(19) United States
(12) Patent Application Publication
(10) Pub. No.: US 2016/0174842 A1
(43) Pub. Date: Jun. 23, 2016

(54) EPIDERMAL ELECTRONICS SYSTEMS HAVING RADIO FREQUENCY ANTENNAS SYSTEMS AND METHODS

(71) Applicant: Elwha LLC, Bellevue, WA (US)

(72) Inventors: Roderick A. Hyde, Redmond, WA (US); Elizabeth A. Sweeney, Seattle, WA (US); Lowell L. Wood, JR., Bellevue, WA (US)

(73) Assignee: ELWHA LLC, Bellevue, WA (US)

(21) Appl. No.: 14/573,097

(22) Filed: Dec. 17, 2014

(51) Int. Cl. A61B 5/00; G01S 13/86; H01Q 3/30; G01S 13/89; A61B 5/11; H01Q 1/27

(52) U.S. Cl. CPC A61B 5/0031; A61B 5/11; A61B 5/1116; A61B 5/6867; H01Q 1/273; H01Q 3/30; G01S 13/89; G01S 13/86; G01S 2013/0254

(57) ABSTRACT

An epidermal electronics device, including a barrier layer configured to attach the epidermal electronics device to the skin of a user, an antenna array coupled to the barrier layer, and a control circuit coupled to the actively phased antenna, wherein the control circuit is configured to actively phase the antenna array.

### BACKGROUND

Epidermal electronic devices are typically flexible devices that conform to the tissue (e.g., skin) of a user. The devices are attached to a surface and can provide data regarding the surface. Generally, they include electronic components secured by a substrate layer. The electronic components may include sensors for measuring parameters related to the surface on which the epidermal electronic device is attached. Epidermal electronic devices may also include components for interacting with the surface to which the epidermal electronic device is attached.

### SUMMARY

One embodiment relates to an epidermal electronics device including a barrier layer configured to attach the epidermal electronics device to the skin of a user, an actively phased antenna array coupled to the barrier layer, a transceiver circuit configured to transmit and receive radio frequency wireless signals using the actively phased antenna array, and a control circuit coupled to the actively phased antenna array and coupled to the transceiver circuit, wherein the control circuit is configured to control the actively phased antenna array.

Another embodiment relates to an epidermal electronics system. The system includes a first epidermal electronics device and a second epidermal electronics device. The first epidermal electronics device includes a first barrier layer configured to attach the first epidermal electronics device to the skin of a user at a first location, a first antenna coupled to the first barrier layer, a first transceiver circuit configured to transmit and receive radio frequency wireless signals using the antenna, and a first control circuit coupled to the first antenna and coupled to the first transceiver circuit. The second epidermal electronics device includes a second barrier layer configured to attach the second epidermal electronics device to the skin of a user at a second location, a second antenna coupled to the second barrier layer, a second transceiver circuit configured to transmit and receive radio frequency wireless signals using the second antenna, and a second control circuit coupled to the second antenna and coupled to the second transceiver circuit. The first epidermal electronics device and the second epidermal electronics device are configured to communicate a first information with one another, and at least one of the first control circuit and the second control circuit is configured to control at least one of the first antenna and the second antenna based on the first information.

Another embodiment relates to a method for communicating with a remote device using an epidermal electronics device. The method includes determining the orientation of the epidermal electronics device using a control circuit of the epidermal electronics device and at least one sensor and actively phasing an antenna array of the epidermal electronics device based on the orientation of the epidermal electronics device using the control circuit. The antenna array is coupled to the control circuit and configured to be controlled by the control circuit.

Another embodiment relates to an epidermal electronics system for sensing the posture of a user. The system includes a first epidermal electronics device and a second epidermal electronics device. The first epidermal electronics device includes a first barrier layer configured to attach the first epidermal electronics device to the skin of a user at a first location, a first antenna coupled to the first barrier layer, a first transceiver circuit configured to transmit and receive radio frequency wireless signals using the antenna, a first position sensor, and a first control circuit coupled to the first antenna, coupled to the first position sensor, and coupled to the first transceiver circuit. The second epidermal electronics device includes a second barrier layer configured to attach the second epidermal electronics device to the skin of a user at a second location, a second antenna coupled to the second barrier layer, a second transceiver circuit configured to transmit and receive radio frequency wireless signals using the second antenna, a second position sensor, and a second control circuit coupled to the second antenna, coupled to the second position sensor, and coupled to the second transceiver circuit. The first epidermal electronics device and the second epidermal electronics device communicate using the first antenna, the second antenna, the first transceiver circuit, and the second transceiver circuit. At least one of the first control circuit and the second control circuit is configured to estimate an orientation of the first epidermal electronics device relative to the second epidermal electronics device.

Another embodiment relates to an epidermal electronics device including a barrier layer configured to attach the epidermal electronics device to the skin of a user, an antenna coupled to the barrier layer, a transceiver circuit configured to transmit and receive radio frequency wireless signals using the antenna and receive radio frequency wireless signals using the antenna, and a control circuit coupled to the antenna and coupled to the transceiver circuit. The control circuit is configured to control the antenna, the radio frequency wireless signals include radar waves, and the control circuit is configured to process the radio frequency wireless signals received by the antenna.

Another embodiment relates to a method for performing personal radar functions for a user using an epidermal electronics device. The method includes receiving radar waves from an object using an antenna of the epidermal electronics device and processing the received radar waves using a control circuit of the epidermal electronics device.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is an illustration of a user wearing an epidermal electronics device according to one embodiment.

FIG. 2A is a schematic view of an epidermal electronics device showing a plurality of layers according to one embodiment.

FIG. 2B is a schematic profile view of an epidermal electronics device showing a plurality of layers and an attachment surface according to one embodiment.

FIG. 2C is a schematic profile view of an epidermal electronics device including an impedance barrier according to one embodiment.

FIG. 3A is schematic block diagram of the components of an epidermal electronics device according to one embodiment.

FIG. 3B is schematic block diagram of the components of an epidermal electronics device including an electronics module according to one embodiment.

FIG. 3B is a schematic illustration of an epidermal electronics device in communication with remote devices according to one embodiment.

FIG. 4 is a schematic illustration of an epidermal electronics device controlling a phased antenna array according to one embodiment.

FIG. 5 is a schematic illustration of an epidermal electronics device functioning as a personal radar system according to one embodiment.

FIG. 6 is a schematic illustration of an epidermal electronics system including a plurality of epidermal electronics devices according to one embodiment.

FIG. 7 illustrates a block diagram for a method of communicating with a remote device using an epidermal electronics device according to one embodiment.

FIG. 8 illustrates a block diagram for a method of providing personal radar functions using an epidermal electronics device worn by a user according to one embodiment.

### DETAILED DESCRIPTION

In the following detailed description, reference is made to the accompanying drawings, which form a part thereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Generally, an epidermal electronics device may include a thin layer of electronic circuits. This thin layer is supported by a barrier layer and optionally encapsulated by a substrate layer. The device is configured to attach to skin or other tissue. The device is also configured to allow the electronic circuits to flex without being damaged. The electronic circuits may be or be circuitry having a serpentine design. The epidermal electronics device can include electronics for measuring various parameters, communication, control, data processing, and/or other functions. A number of embodiments of epidermal electronics devices are described in “Flexible and Stretchable Electronic Systems for Epidermal Electronics”, U.S. patent application Ser. No. 13/492,636 by John A. Rogers and Dae-Hyeong Kim. Additional embodiments of epidermal electronics devices are described in “Multifunctional Epidermal Electronics Printed Directly Onto the Skin”, Yeo et. al, Advanced Materials (2013).

Referring to the figures generally, various embodiments disclosed herein relate to epidermal electronics devices, and more specifically, epidermal electronics devices including radio frequency (RF) transmitters, receivers, transceivers, and/or one or more antennas. Epidermal electronics devices including RF transmitters, receivers, and/or transceivers may be used for applications including communications, posture estimation, radar systems, and/or other applications. For example, an epidermal electronics device may be used as an antenna or antenna array to communicate with remote devices. As described in greater detail herein, one or more epidermal electronics devices may be used as antennas for communication with a cell phone tower. The epidermal electronics device(s) may also be in communication with a cell phone or other mobile electronics device used by a user wearing the epidermal electronics device(s). The epidermal electronics devices may enhance communication between the cell phone and the cell tower in comparison to the cell phone alone.

In some embodiments, a plurality of epidermal electronics devices (e.g., one or more) worn by a single user communicate with each other using one or more antennas and/or other radio frequency wireless communication hardware (e.g., transceivers). The epidermal electronics devices can communicate information including information related to the position and/or orientation of each epidermal electronics device. Using this information (e.g., the position and orientation of a plurality of epidermal electronics devices), the posture of a user can be determined.

In some embodiments, one or more epidermal electronic devices are used as a radar system. The epidermal electronics device may emit radar waves via an antenna and receive radar waves reflected from other objects. Using a control circuit or other processing hardware included in the epidermal electronics device or elsewhere, information about the object can be determined (e.g., position, shape, movement speed, movement direction, etc.). Using this information, the epidermal electronics device may provide several radar based functions such as collision warnings, object imaging, object monitoring (e.g., the speed, direction, etc. of the object), and/or other radar based functions.

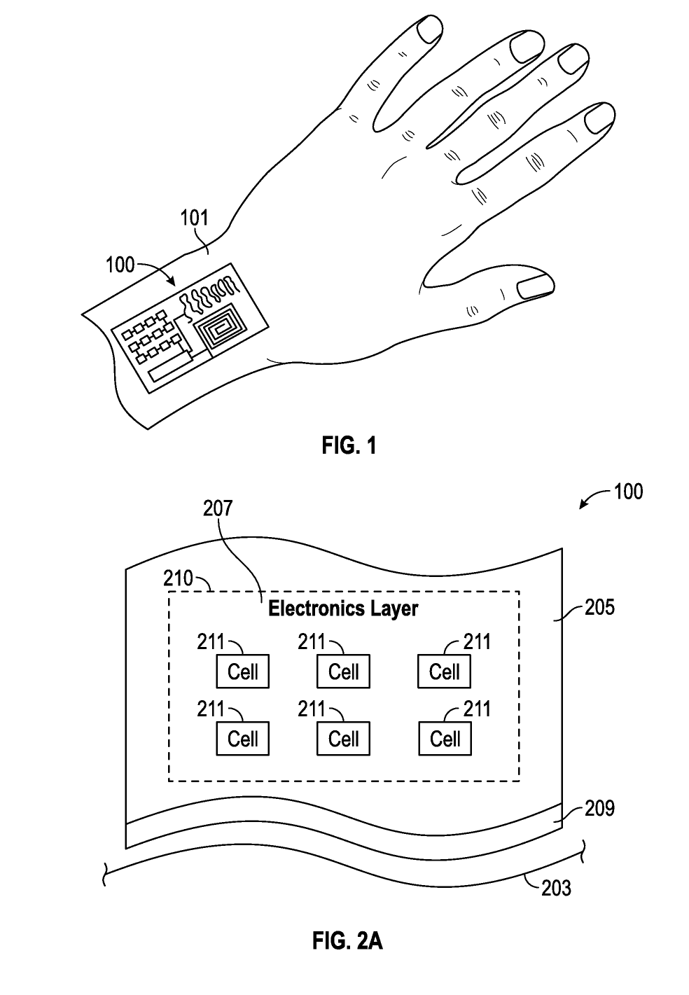

Referring now to FIG. 1, epidermal electronics device 100 is shown according to one embodiment. Epidermal electronics device 100 is worn by a user 101. Epidermal electronics device 100 may be worn on the skin of user 101 as illustrated. For example, epidermal electronics device 100 may be attached to the skin of user 101. Epidermal electronics device 100 may be worn on an arm, hand, leg, foot, torso, head, or other body part of user 101. In some embodiments, described in greater detail herein, a system of a plurality of epidermal electronics devices 100 are worn by user 101.

Epidermal electronics device 100 provides the enhanced communications and other benefits described herein while worn by user 101. Advantageously this may be used in embodiments where epidermal electronics device 100 is worn on the skin of user 101. This is in contrast to devices which are embedded in garments of user 101 or otherwise carried by user 101. Moving a device between garments is not necessary to receive the benefits of the device as epidermal electronics device 100 is worn by user 101. In other words, epidermal electronics device 100 remains with user 101, attached to the skin of user 101, regardless of the clothing worn by user 101. Therefore, user 101 may more easily use a system including one or more epidermal electronics devices 100 without transferring epidermal electronics device 100 between garments. Epidermal electronics device 100 can be worn on the skin. This allows user 101 to use epidermal electronics device 100 frequently without requiring one epidermal electronics device 100 for each garment worn by user 101 or requiring user 101 to move or carry a device with himself or herself.

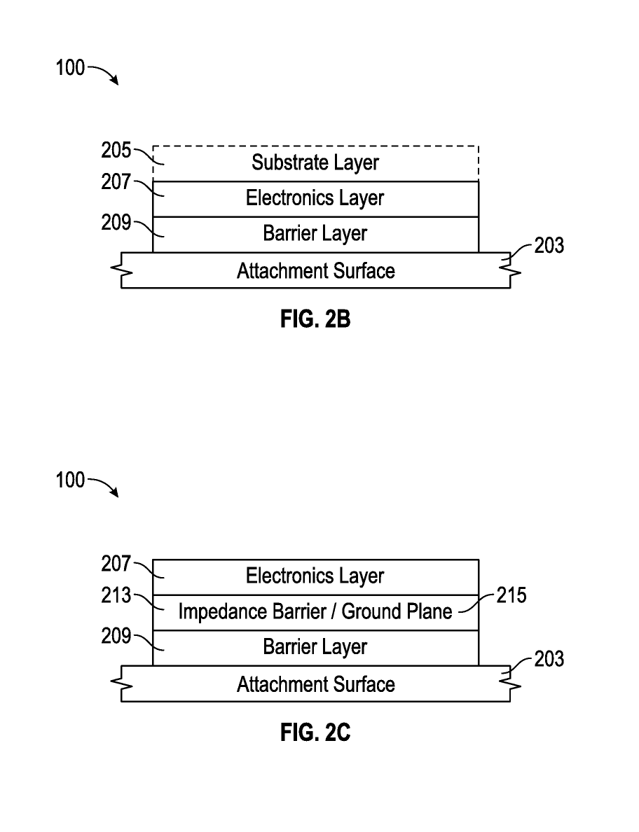

Referring to FIG. 2A, an embodiment of epidermal electronics device 100 is shown to include a plurality of layers. Epidermal electronics device 100 may include substrate layer 205. Substrate layer 205 provides mechanical support to other components of epidermal electronics device 100 in embodiments of epidermal electronics device 100 including substrate layer 205. Epidermal electronics device 100 further includes electronics layer 207, which may be located between substrate layer 205 and barrier layer 209. Electronics layer 207 is shown through substrate layer 205 with view 210. Electronics layer 207 may include the electronic components of epidermal electronics device 100. In some embodiments, one or more electronic components of epidermal electronics device 100 are included in a plurality of cells 211. Cells 211 may be interconnected and allow for communication between a plurality of electronic components. Barrier layer 209 may provide protection to these components. Epidermal electronics device 100 is illustrated as attached to attachment surface 203.

Substrate layer 205 may facilitate the transfer of epidermal electronics device 100 to attachment surface 203. For example, substrate layer 205 may provide a backing which is used to transfer electronics layer 207 to attachment surface 203. Substrate layer 205 may then peel away from electronics layer 207 leaving electronics layer 207 attached to attachment surface 203. Substrate layer 205 may also provide protection to electronics layer 207 during the handling of epidermal electronics device 100. Substrate layer 205 may further provide mechanical support for electronics layer 207. Substrate layer 205 can be an elastomer or polymer suited for use in contact with organic tissue. In some embodiments, the substrate layer 205 is a biocompatible or otherwise inert material. For example, the substrate layer 205 may be a rubber or silicone material. In some embodiments, substrate layer 205 is water soluble. Substrate layer 205 may be dissolved following transfer of the epidermal electronics device 100 onto the attachment surface 203. In some embodiments, substrate layer 205 need not be biocompatible as it is removed completely or partially following the transfer of epidermal electronics device 100 onto the attachment surface 203. Substrate layer 205 may also provide protection to electronics layer 207 from damage that may otherwise result from moisture, physical damage, electrical interference, magnetic interference, etc. In alternative embodiments, epidermal electronics device 100 does not include substrate layer 205.

Attachment surface 203 is the surface to which epidermal electronics device 100 is attached. In one embodiment, attachment surface 203 is the skin of a user. Epidermal electronics device 100 is held in contact with attachment surface 203 through conformal contact. In some embodiments, epidermal electronics device 100 is held in contact with attachment surface 203 through close-contact atomic forces or van der Waals interactions. In other embodiments, epidermal electronics device 100 is held in contact with attachment surface 203 through the use of an adhesive. The adhesive may be applied after the epidermal electronics device 100 is placed on attachment surface 203. For example, the adhesive may be a spray on bandage or may be adhesive tape. The adhesive may also be included as a component of barrier layer 209. In other embodiments, epidermal electronics device 100 is attached to other attachment surfaces 103. For example, attachment surface 203 may be clothing worn by the user, a device worn by the user, and/or another surface associated with a user.

Barrier layer 209 provides protection to one or more components within epidermal electronics device 100. In some embodiments (e.g., embodiments in which epidermal electronics device 100 does not include substrate layer 205), barrier layer 209 may provide mechanical support for electronics layer 207. According to one embodiment, barrier layer 209 at least partially encompasses the electronics layer 207. Barrier layer 209 provides protection to electronics layer 207 from external sources of damage. External sources of damage may include moisture, physical damage (e.g., from a patient touching epidermal electronics device 100), electrical interference, magnetic interference, etc. In some embodiments, barrier layer 209 encompasses the entirety of epidermal electronics layer 207. In other embodiments, barrier layer 209 only coats electronics layer 207 on the surface opposite substrate layer 205. Barrier layer 209 may also partially coat electronics layer 207 to allow for contact between elements or cells 211 of electronics layer 207 and the attachment surface 203.

With continued reference to FIG. 2A, electronics layer 207 may be located between substrate layer 205 and barrier layer 209. Substrate layer 205 provides support for the elements of electronics layer 207. View 210, illustrated as a dashed line, shows electronics layer 207 through substrate layer 205. In one embodiment, electronics layer 207 includes an array of cells 211. Cells 211 contain individual sensors or components. Cells 211 are also in communication with other components in electronics layer 207. In some embodiments, cells 211 may be in communication with each other or a subset of other cells 211 within epidermal electronics device 100. Cells 211 may also be in communication with other elements. For example, cells 211 may be in communication with a power supply, control circuit, and/or communications device. Cells 211 may also contain connections to allow power delivery to the component in the cell, input/output to and from the component in the cell, and/or multiplexing circuitry. In some embodiments, cells 211 may contain sensors such as accelerometers, inclinometers, or gyroscopes. These sensors may be of the micro electro-mechanical systems (MEMS) type given the small scale of epidermal electronics device 100 and associated components. The sensors may also be part of or supported by integrated circuits or systems on a chip (SOCs). Cells 211 may also contain interaction devices such as drug delivery systems, electrodes, motion capture markers, etc. The interaction devices may also be MEMS, part of or supported by integrated circuits, or SOCs. According to various alternative embodiments, cells 211 include circuitry facilitating multiplexing of sensor output, transformers, amplifiers, circuitry for processing data and control signals, one or more transistors, etc.

In one embodiment, cells 211 include one or more RF antennas. RF antennas used to receive electromagnetic radiation may be considered to be a sensor 315 and or interaction device 317 as the terms are used herein. RF antennas can be used by epidermal electronics device 100 to communicate with remote devices (e.g., devices other than epidermal electronics device 100). For example, epidermal electronics device 100 may communicate using radio waves and one or more RF antennas with other epidermal electronics devices 100, RF transmitters, RF receivers, RF transceivers (e.g., cell tower included in a cellular communications network), mobile communications devices (e.g., cell phone, tablet, etc.), personal computers or peripherals (e.g., desktop computer, printer, etc.), wireless home devices (e.g., smart locks, appliances, light switches, or other wireless home automation devices), communication network devices (e.g., wireless router, cellular voice transceiver, cellular data transceiver, cell tower, etc.), and/or other remote devices which are configured to transmit and/or receive radio frequency transmissions.

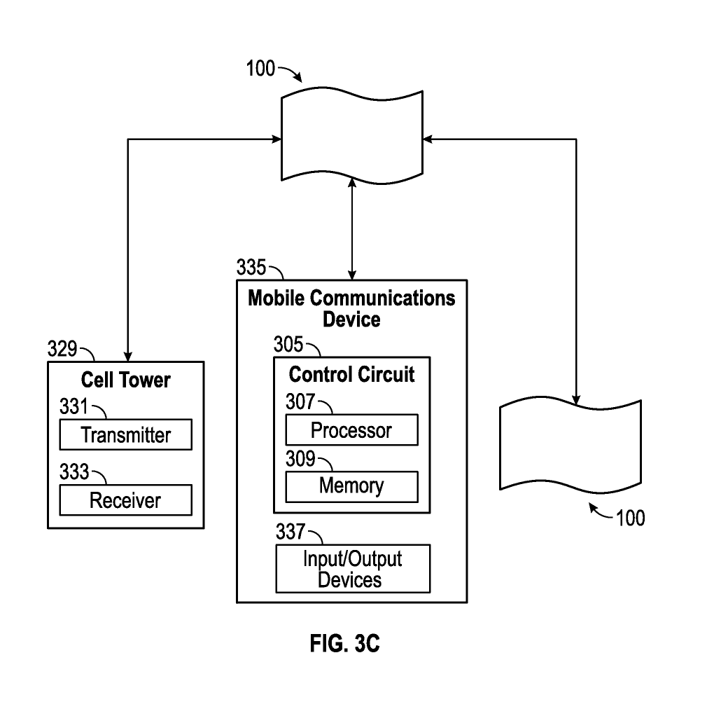

In some embodiments, RF antennas can be used to transmit and/or receive radio frequency transmissions in one or more radar spectrums. Epidermal electronics device 100 can use the emitted radar waves of one or more RF antennas and reflected radar waves from objects to detect the objects. Epidermal electronics device 100 can determine the proximity to objects, the trajectory of objects, the speed of objects, and/or otherwise function as a radar system. Advantageously, epidermal electronics device 100 can act as a personal radar system for user 101. The use of RF antennas for communication and/or radar functions is described in greater detail herein with respect to FIGS. 4-8.

Referring now to FIG. 2B a cross section schematic view of epidermal electronics device 100 is shown according to one embodiment. Substrate layer 205 is the topmost layer relative to attachment surface 203. Barrier layer 209 is in contact with attachment surface 203 and protects electronics layer 207. Electronics layer 207 is between barrier layer 209 and substrate layer 205. In some embodiments, epidermal electronics device 100 further includes one or more local impedance references.

Substrate layer 205 may provide physical support for electronics layer 207. Substrate layer 205 may also facilitate attachment of the epidermal electronics device 100, including electronics layer 207 and barrier layer 209, to the attachment surface 203. In some embodiments, substrate layer 205 is discarded or dissolved after epidermal electronics device 100 has been attached to attachment surface 203.

Electronics layer 207 is illustrated as including components on a layer of material. The layer of material may be used to provide mechanical support to the components of electronics layer 207. It may also be used to facilitate manufacturing of electronics layer 207. In some embodiments, electronics layer 207 is made up only of the electronic components therein (e.g., there is no supporting layer of material). In such a case, electronics layer 207 may be manufactured on a substrate which provides the mechanical support necessary to make and use epidermal electronics device 100.

Barrier layer 209 provides protection to the components of electronics layer 207. Barrier layer 209 may prevent external forces and elements from interfering with the functions of electronics layer 207. For example, barrier layer 209 may prevent moisture from reaching electronics layer 207. In some embodiments, barrier layer 209 may also prevent physical damage to the components of electronics layer 207. Barrier layer 209 may also shield electronics layer 207 from outside sources of radiation, magnetic fields, light, etc. In some embodiments, barrier layer 209 is permeable or semipermeable.

In alternative embodiments, epidermal electronics device 100 may include a subset of the layers described above. For example, epidermal electronics device 100 may include only barrier layer 209 and the electronic components described herein. Barrier layer 209 may protect the electronic components, attach epidermal electronics device 100 to attachment surface 203, and provide a surface on which epidermal electronics device 100 is constructed. Substrate layer 205 is an optional component of epidermal electronics device 100.

Referring now to FIG. 2C, a cross section schematic view of epidermal electronics device 100 including a local impedance reference (e.g., impedance barrier 213) is shown according to one embodiment. Epidermal electronics device 100 can include a local impedance reference to compensate for factors which may adversely impact performance of one or more RF antennas included in epidermal electronics device 100. For example, the performance of one or more RF antennas may be impacted by the thinness of the RF antenna included in the epidermal electronics device. The thinness of the RF antenna may result in the RF antenna having a high capacitance. Additionally, attachment surface 203 (e.g., the skin of user 101) may have variable impedance (e.g., the impedance of the skin of user 101 may vary across the surface of the skin). Advantageously, the inclusion of a local impedance reference between the RF antenna and attachment surface 203 may improve performance of one or more RF antennas.

In one embodiment, epidermal electronics device 100 includes impedance barrier 213. Impedance barrier 213 may be a material with uniform impedance. Therefore, impedance barrier 213 may function as a local impedance reference for one or more RF antennas. In some embodiments, impedance barrier 213 is a material with high impedance. In some embodiments, impedance barrier 213 is a material with specified dielectric constant at an RF frequency used by an RF antenna. For example, impedance barrier may be or include materials such as ceramics, dielectrics, polymers, organic materials, or other materials suitable for use as an impedance barrier.

Impedance barrier 213 can be a separate layer positioned between an RF antenna and attachment surface 203. For example, impedance barrier 213 can be positioned between barrier layer 209 and electronics layer 207. In alternative embodiments, impedance barrier 213 may be located in other positions. For example, impedance barrier 213 can be positioned between attachment surface 203 and barrier layer 209. In further embodiments, barrier layer 209 may be or include impedance barrier 213. Alternatively, electronics layer 207 may include impedance barrier 213 such that impedance barrier 213 is located between at least one RF antenna and attachment surface 203.

In one embodiment, epidermal electronics device 100 includes ground plane 215 positioned between at least one RF antenna and attachment surface 203. Ground plane 215 may function as a local impedance reference for use with one or more RF antennas. Ground plane 215 can be an electrically conductive surface configured to operate as a ground plane. For example, ground plane 215 may be a section of copper foil. In some embodiments, ground plane 215 is configured to be at least one quarter the size of the radio frequency wavelength used by the RF antenna. Ground plane 215 need not be connected to ground.

In some embodiments, ground plane 215 is a continuous surface (e.g., flat copper foil). In alternative embodiments, ground plane 215 is not a continuous surface. For example, ground plane 215 may include a plurality of conductive wires extending from the base of an RF antenna.

Ground plane 215 can be a separate layer positioned between an RF antenna and attachment surface 203. For example, ground plane 215 can be positioned between barrier layer 209 and electronics layer 207. In alternative embodiments, ground plane 215 may be located in other positions. For example, ground plane 215 can be between attachment surface 203 and barrier layer 209. In further embodiments, barrier layer 209 may be or include ground plane 215. Alternatively, electronics layer 207 may include ground plane 215 such that ground plane 215 is located between at least one RF antenna and attachment surface 203. In some embodiments, epidermal electronics device 100 includes both impedance barrier 213 and ground plane 215. For example, impedance barrier 213 can be located between at least one RF antenna and ground plane 215, which is located above attachment surface 203. For example, impedance barrier 213 can serve as a dielectric spacer within a microstrip antenna (e.g., placing the dielectric layer between an RF antenna top layer and a ground plane bottom layer).

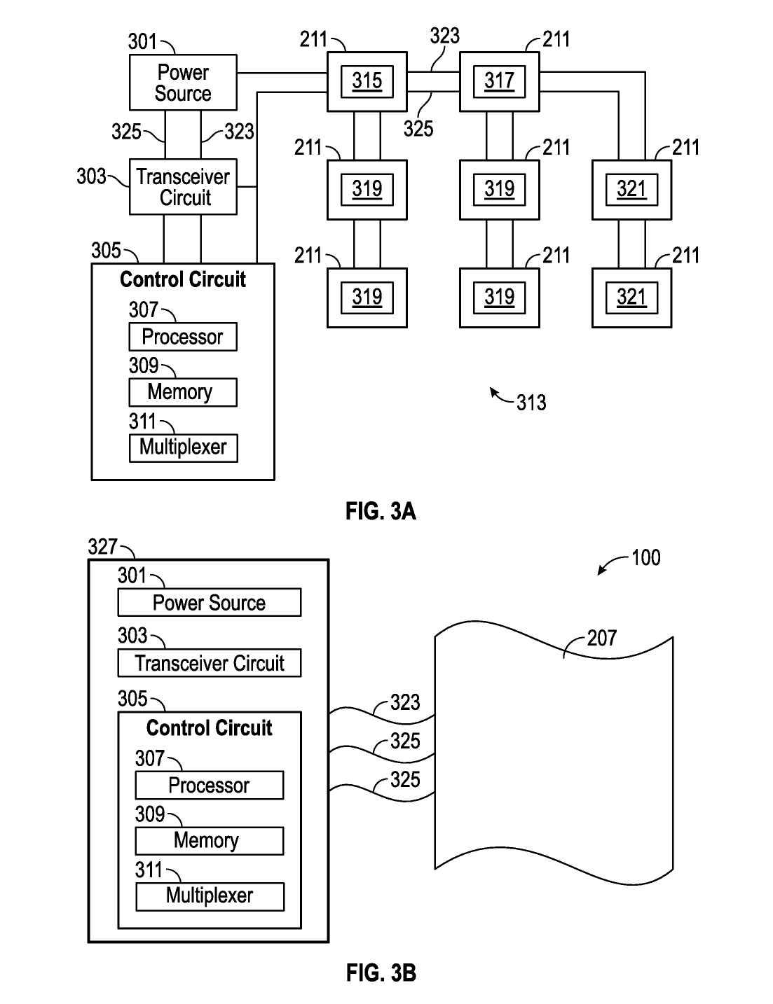

Referring now to FIG. 3A, various electronic components of epidermal electronics device 100 are shown according to one embodiment. The electronic components of epidermal electronics device 100 may be included in electronics assembly 313. Electronics assembly 313 includes components which are located in electronics layer 207. As depicted, electronics assembly 313 and the components therein may not include an additional layer of material (e.g., electronics assembly 313 may include only circuits and components without a supporting material or substrate). In some embodiments, electronics assembly 313 is produced on substrate layer 205 (not pictured in FIG. 3A). Electronics assembly 313 may include cells 211, sensors 315, interaction devices 317, power source 301 connected to other components via power connection 323, transceiver circuit 303, control circuit 305, and input/output connection 325. In some embodiments, control circuit 305 further includes memory 309, processor 307, and multiplexer 311.

Transceiver circuit 303 may be included in electronics assembly 313. Transceiver circuit 303 provides data transfer to and from epidermal electronics device 100 by transmitting and/or receiving transmissions. In one embodiment, transceiver circuit 303 receives and/or transmits RF transmissions using one or more RF antennas 319. In some embodiments, one RF antenna 319 may be used to receive a RF transmission while a second RF antenna 319 is used to transmit an RF transmission. In further embodiments, transceiver circuit 303 may include components to allow for the transmission and/or reception of other transmissions. For example, transceiver circuit 303 may include a light source and light detector for use in transmitting and/or receiving optical transmissions. Alternatively or additionally, transceiver circuit 303 may include a microphone and/or speaker for use in transmitting and/or receiving audio transmissions.

Transceiver circuit 303 may further include hardware for the reception, generation, manipulation of, and/or further processing of transmissions or signals. For example, transceiver circuit 303 may include mixers, filters, a modulator, a demodulator, and/or other components for processing signals.

Transceiver circuit 303 and/or RF antennas 319 allow for wireless communication between epidermal electronics device 100 and one or more remote devices. A variety of wireless communications technique or protocols may be used. For example, epidermal electronics device may communicate using a wireless network or wireless point to point communication and using one or more protocols such as WiFi, Zigbee, Bluetooth, CDMA, GSM, or other communications protocols (e.g., infrared protocols, optical protocols, ultrasound protocols, etc.).

In one embodiment, transceiver circuit 303 uses one or more RF antennas 319 to transmit and/or receive communications signals. Transceiver circuit 303 may generate a communications signal, send the signal to one or more RF antennas 319 included in epidermal electronics device 100, and transmit the signals through the one or more RF antennas 319.

One or more RF antennas 319 can receive a communications signal. The communications signal may be sent to transceiver circuit 303 via the one or more RF antennas 319. Transceiver circuit 303 can then process the communications signal. The processed communications signal may be passed to control circuit 305 from transceiver circuit 303. When sending transmission or communications signals, control circuit 305 may cause transceiver circuit 303 to format a signal and transmit it using RF antennas 319. For example, control circuit 305 may cause transceiver circuit 303 to modulate a signal for transmission such that the signal contains information stored in or received at control circuit 305.

Power connection 323 transfers power from power source 301 to other components in electronics layer 207. Power connection 323 provides power from power source 301 to transceiver circuit 303, control circuit 305, cells 211, and the components within cells 211 such as interaction devices 317 and sensors 315. Power connection 323 may be a wired or wireless connection. Power connection 323 may be a conductive wire (e.g., copper, aluminum, etc.). Power connection 323 may be a semiconductor. Where power connection 323 is a wired connection, power connection 323 is configured to maintain mechanical integrity when components of electronics layer 207 move relative to one another. For example, power connection 323 may be a length of wire long enough to allow movement of the components without causing deformation of power connection 323 sufficient to break the connection. Power connection 323 may also be a wireless connection for delivering power (e.g., direct induction, resonant magnetic induction, etc.).

Power source 301 provides electrical power to components within electronics layer 207. In one embodiment, power source 301 is a battery. For example, power source 301 may be a disposable battery, rechargeable battery, and/or removable battery. In some embodiments, power source 301 is configured to allow recharging of power source 301 without removing power source 301 from electronics layer 207. For example, power source 301 may be a rechargeable battery configured to be recharged through wireless changing (e.g., inductive charging). In other embodiments, power source 301 is configured to receive direct current from a source outside the electronics layer 207. In further embodiments, power source 301 is configured to receive alternating current from a source outside the electronics layer 207. Power source 301 may include a transformer. In some embodiments, power source 301 is configured to receive power from a wireless source (e.g., such that power source 301 is a coil configured to receive power through induction). According to various alternative embodiments, power source 301 is a capacitor configured to be charged by a wired or wireless source, one or more solar cells, or a metamaterial configured to provide power via microwaves.

Epidermal electronics device 100 may be powered in whole or in part by electrical power generated by one or more power scavenging devices 321 included in epidermal electronics device 100. For example, power scavenging device 321 can be configured to generate power from solar energy, transmitted energy (e.g., through inductive charging), and/or mechanical sources of energy such as the movement of user 101. Power scavenging device 321 may include hardware for generating energy from the above described sources. For example, power scavenging device 321 may be or include a solar cell, inductive coil (e.g., for wireless charging), a self-winding mechanism, and/or other power generating hardware.

In some embodiments, epidermal electronics device 100 is powered, in whole or in part, by deformation of epidermal electronics device caused by the movement of user 101. In one embodiment, power scavenging device 321 is or includes a deformable capacitor. As power scavenging device 321 and the deformable capacitor deform due to the movement of user 101 (e.g., caused by the flexing of muscle), power scavenging device 321 can act as a charge-pump. Muscle movement can cause the stretching and/or compression of either electric and/or magnetic field lines. Longitudinal stretching and/or transverse compression generates electrical power. Power scavenging device 321 collects this energy and uses it to power epidermal electronics device 100. In other embodiments, electrical energy is applied to power scavenging device 321 in order to cause the deformation of epidermal electronics device 100. This deformation may cause epidermal electronics device 100 to act as artificial muscle. In other embodiments, power scavenging device 321 includes one or more piezoelectric systems configured to generate electricity from the movement or deformation of epidermal electronics device 100 in response to movement of user 101.

With continued reference to FIG. 3A, input/output connection 325 may be a wire connection between cell 211 and control circuit 305. Input/output connection 325 may be configured to allow the connection to flex and deform without suffering mechanical failure. In such a case, input/output connection 325 is configured to maintain the connection between cell 211 and control circuit 305 during deformation of epidermal electronics device 100 due to movement of attachment surface 203. In some embodiments, input/output connection 325 allows for deformation while maintaining mechanical integrity by including an additional length of wire which allows for connection points to separate from one another. For example, input/output connection 325 may be a wire with slack to allow two or more components to move relative to one another and not cause mechanical degradation of the input/output connection. In some embodiments, input/output connection 325 is a conductive wire (e.g., copper, aluminum, etc.). Input/output connection 325 may be a semiconductor. In some embodiments, input/output connection 325 is a wireless connection.

Input/output connection 325 allows the components within cell 211 to communicate data to control circuit 305. The component within cell 211 may output data to the control circuit through input/output connection 325. For example, sensor 315 located in cell 211 may output measurement data, in the form of a voltage, across input/output connection 325 to control circuit 305. Input/output connection 325 also allows for the control circuit to communicate with the component within cell 211. Control circuit 305 may send an input to a component within cell 211 through input/output connection 325. For example, control circuit 305 may send an input signal to interaction device 317. Cell 211 may also facilitate communication. Control circuit 305 may also send a calibration signal to sensor 315 or interaction device 317 using input/output connection 325. In some embodiments, power connection 323 and input/output connection 325 are integrated into a single connection. For example, an integrated connection may provide power and input/output through a modulated or otherwise alterable signal. Input/output connection 325 may allow for transmissions and/or signals received by one or more RF antennas 319 to be communicated to or received at transceiver circuit 303 and/or control circuit 305. Input/output connection 325 may further allow for signals generated by transceiver circuit 303 and/or control circuit 305 to be transmitted by one or more RF antennas 319.

Control circuit 305 controls the electronic components of epidermal electronics device 100. Control circuit 305 may contain circuitry, hardware, and/or software for facilitating and/or performing the functions of epidermal electronics device 100 described herein. Control circuit 305 may handle inputs, process inputs, run programs, handle instructions, route information, control memory, control a processor, process data, generate outputs, communicate with other devices or hardware, and/or otherwise perform general or specific computing tasks.

Control circuit 305 may further include multiplexer 311, processor 307, and/or memory 309. Processor 307 may be implemented as a general-purpose processor, an application specific integrated circuit (ASIC), one or more field programmable gate arrays (FPGAs), a digital-signal-processor (DSP), a group of processing components, or other suitable electronic processing components. Memory 309 is one or more devices (e.g., RAM, ROM, Flash Memory, hard disk storage, etc.) for storing data and/or computer code for facilitating the various processes described herein. Memory 309 may be or include non-transient volatile memory or non-volatile memory. Memory 309 may include database components, object code components, script components, or any other type of information structure for supporting the various activities and information structures described herein. Memory 309 may be communicably connected to processor 307 and provide computer code or instructions to processor 307 for executing the processes described herein. Multiplexer 311 may be configured to allow multiple sensors 315, RF antennas 319, and/or interaction devices 317 to share an input/output connection 325. In some embodiments, cells 211 also facilitate multiplexing of signals from multiple components.

Memory 309 can include one or more modules for facilitating the functions of epidermal electronics device 100 described herein. Modules may be or include modules for communicating with remote devices using transceiver circuit 303 and RF antennas 319, phasing RF antennas 319, estimating the orientation of epidermal electronics device 100 using sensors 315, providing haptic feedback to user 101 using interaction device 317, controlling radar functions of epidermal electronics device 100, and/or performing other functions of epidermal electronics device 100. Modules may be executed by processor 307 to facilitate the functions described herein. For example, a module may be used to receive communications from a mobile communications device such as a cell phone using RF antenna 319 and transceiver circuit 303. The module may also control transceiver circuit 303 and RF antenna 319 such that the transmission received from the mobile communications device is transmitted to a cell tower or other network receiver or transceiver.

Continuing the example, in one embodiment control circuit 305 executes a module in memory 309 to cause a phased transmission and/or reception using a plurality of RF antennas 319. Control circuit 305 controls the plurality of RF antennas 319 as a phased array. In an embodiment, control circuit 305 can actively phase an array of RF antennas 319 by applying a time shift between transmission of a signal from one individual antenna 319 of the antenna array and transmission of a signal from another individual antenna 319 of the antenna array. This allows for directed transmissions to be sent to a receiver or transceiver. In an embodiment, control circuit 305 can actively phase an array of RF antennas 319 by applying a time shift between delivery of a signal received from one individual antenna 319 of the antenna array and delivery of a signal received from another individual antenna 319 of the antenna array. This allows for directed reception of signals from a receiver or transceiver. Control circuit 305 may phase the array of RF antennas 319 to compensate for the orientation and/or position of epidermal electronics device 100. For example, epidermal electronics device 100 may be attached to an arm of user 101 who is located in the center of a room, and whose arm oriented towards one wall and is tilted downwards by 30 degrees. Based on this position and orientation, control circuit 305 may phase the array of RF antennas 319 to transmit towards a WiFi base station in the ceiling of the room near one corner. For another example, the shape of epidermal electronics device 100 may be curved (e.g., on a curved arm of user 101) and control circuit 305 may cause transceiver circuit 303 to phase a transmission using a plurality of RF antennas 319. This results in the array of RF antennas 319 approximating a flat antenna or otherwise causes a steered or directed transmission which accounts for the curved orientation of epidermal electronics device 100.

Control circuit 305 may execute a module stored in memory 309 in order to determine or estimate the position and/or orientation of epidermal electronics device 100. Control circuit 305 may process inputs from one or more sensors 315 according to a program in the module in order to estimate the position and/or orientation of epidermal electronics device 100. For example, inputs from sensors 315 such as accelerometers, inclinometers, strain sensors, magnetometers, cameras, gyroscopes, etc. may be processed to determine orientation. For example, inputs from sensors 315 such as accelerometers, GPS receivers, cameras, etc. may be processed to determine position. Control circuit 305 may further determine the position and/or orientation of one epidermal electronics device 100 relative to another epidermal electronics device 100 based on information (e.g., sensor 315 data received from other epidermal electronics device 100). Control circuit 305 may further determine the posture of user 101 based on position and/or orientation and/or other data from a plurality of epidermal electronics device worn by user 101. The position and/or orientation may be used in phasing a plurality of RF antennas 319 as described herein and/or for other applications.

Control circuit 305 may also cause the transmission of radar waves from one or more RF antennas 319 and/or process return signals received by RF antennas 319 in conjunction with transceiver circuit 303. Control circuit 305 may send, receive, and/or process radar waves according to a program or module stored in memory 309. Control circuit 305 may perform a variety of functions according to one or more modules such as imaging an object using radar techniques, determining the distance the object is from epidermal electronics device 100 using radar techniques, determining the speed and/or trajectory of an object using radar techniques, and/or otherwise providing information about objects remote from epidermal electronics device 100.

Continuing the example, control circuit 305 may provide reports to user 101, feedback to user 101, and/or otherwise communicate with user 101 according to a module in memory 309 and using interaction device 317. For example, control circuit 305 may cause interaction device 317 to produce a vibration and/or noise to warn a user of an impending impact with an object. The object may be detected and the impending impact estimated using radar based techniques such as those described herein.

Memory 309, processor 307, and/or other components of control circuit 305 may facilitate these and/or other functions of epidermal electronics device 100 described herein using one or more programming techniques, data manipulation techniques, and/or processing techniques such as using algorithms, routines, lookup tables, arrays, searching, databases, comparisons, instructions, etc.

In some embodiments, the functions of control circuit 305 are carried out by the circuitry of cells 211. For example, cells 211 may include transistors and/or additional components which allow cell 211 or a network of cells 211 to perform the above described functions of control circuit 305. In other embodiments, control circuit 305 is located in an area not within electronics layer 207. In one embodiment, transceiver circuit 303 may send and receive control signals and data. For example, an external control circuit 305 may perform the above described functions with transceiver circuit 303 relaying data between the components of the electronics layer 207 (e.g., sensors 315 and interaction devices 317) and the external control circuit 305. For example, control circuit 305 may be located in a mobile communications device and be used to control epidermal electronics device 100.

As previously discussed, epidermal electronics device 100 may include one or more sensors 315. Sensors 315 may be or include any sensor for measuring, sensing, or otherwise generating data regarding epidermal electronics device 100, a remote device, the environment in which epidermal electronics device 100 is present, attachment surface 203, or data otherwise pertinent to the operation of epidermal electronics device 100 as described herein.

In some embodiments, sensors 315 are or include sensors for generating information regarding the orientation, movement, position, location, kinematics, and/or other data related to a state of epidermal electronics device 100. For example, sensors 315 may include accelerometers, gyroscopes, inclinometers, strain sensors, stress sensors, strain gauges, magnetometers, cameras, and/or other sensors for measuring the orientation, location, movement or other related parameters of epidermal electronics device 100.

Sensors 315 may provide data and/or information related to these parameters to control circuit 305. Using this information (e.g., accelerometer measurements, inclinometer measurements, etc.), control circuit 305 may determine, using one or more algorithms, programs, or other techniques, information regarding the physical state of epidermal electronics device 100. As previously discussed, control circuit 305 may determine such information related to epidermal electronics device 100 as location, orientation, position, etc.

In further embodiments, epidermal electronics device 100 may determine information such as the location or position of epidermal electronics device 100 relative to other epidermal electronics devices 100, the orientation of epidermal electronics device 100 relative to other epidermal electronics devices 100, and/or other information relative to other epidermal electronics device 100. Epidermal electronics device 100 may be in communication with additional (e.g., other) epidermal electronics devices 100 (e.g., using RF antenna 319, transceiver circuit 303, and/or control circuit 305). Epidermal electronics device 100 may receive position data, location data, orientation data, and/or other information from the other epidermal electronics devices 100. Using this information, epidermal electronics device 100 can determine its position, location, orientation, etc. relative to the other epidermal electronics devices 100. For example, control circuit 305 may process the information from the other epidermal electronics devices 100 as well as information from local sensors 315 using one or more algorithms, programs, or other techniques to estimate or determine information about epidermal electronics device 100 relative to other epidermal electronics devices 100.

In one embodiment, the relative position or orientation of two (or more) separate epidermal electronics devices 100 can be determined by transmitting a signal from RF antenna 319 within one epidermal device and receiving it with that of another epidermal electronics device, detecting time, phase, or polarization shifts between the transmitted and received signals. In an embodiment, the transmission and reception can be from single RF antennas within each of the epidermal electronics devices. In another embodiment, either the transmission or reception can be performed using signals from individual RF antennas within each epidermal electronics device; for example, by using 3 individual RF antennas within each of the epidermal electronics devices, the relative strengths and phase shifts and time shifts from 9 signals can be compared in order to determine the relative position or orientation of the two epidermal electronics devices.

When transmitting signals, data, or information from a second epidermal electronics device to a first epidermal electronics device so as to determine their relative position and/or orientation, the timing of the transmissions can be controlled. In an embodiment, a control circuit of the second epidermal electronics device can be configured to transmit data based on at least one of a time, a time interval from a previous transmission of data, a position of the second epidermal electronics device, an orientation of the second epidermal electronics device, a change in position of the second epidermal electronics device, a change in orientation of the second epidermal electronics device, and a motion of the second epidermal electronics device, a relative position between the first epidermal electronics device and the second epidermal electronics device, a relative orientation between the first epidermal electronics device and the second epidermal electronics device, and a relative motion between the first epidermal electronics device and the second epidermal electronics device. In another embodiment, the second epidermal electronics device can transmit data based upon receiving a request for the data from the first epidermal electronics device. In an embodiment, a control circuit of the first epidermal electronics device can be configured to transmit the request based on at least one of a time, a time interval from a previous reception of data, a time interval from a previous request for data, a position of the first epidermal electronics device, an orientation of the first epidermal electronics device, a change in position of the first epidermal electronics device, a change in orientation of the first epidermal electronics device, and a motion of the first epidermal electronics device, a relative position between the first epidermal electronics device and the second epidermal electronics device, a relative orientation between the first epidermal electronics device and the second epidermal electronics device, and a relative motion between the first epidermal electronics device and the second epidermal electronics device.

In further embodiments, sensors 315 may be or include sensors for determining other information about the environment in which epidermal electronics device 100 is used. For example, sensors 315 may include sensors such as temperature sensors, moisture sensors, antennas (e.g., to sense one or more fields such as magnetic fields, RF fields from a field source, etc.), light sensors, and/or other sensors. Sensors 315 may include one or more RF antennas 319.

In some embodiments, epidermal electronics device 100 includes one or more interaction devices 317 such as those previously discussed. Interaction device 317 may be or include a device configured to communicate with user 101 (e.g., provide haptic feedback to user 101). For example, interaction device 100 may be or include a vibration motor, an electrode, a light source such as a light emitting diode (LED), a speaker or other acoustic source, and/or other hardware or devices for communicating with user 101. In further embodiments, interaction device 317 is or includes communication hardware for communicating with a remote device. For example, interaction device 317 may be or include a wireless transmitter or transceiver for communicating with remote devices such as mobile communications devices (e.g., a cell phone, wearable computer, etc.). Alternatively, epidermal electronics device 100 may use control circuit 305, transceiver circuit 303, and/or RF antenna 319 to communicate with the remote device (e.g., mobile communication device). Control circuit 305 may control communication with user 101. In some embodiments, a user can customize epidermal electronics device 100 to specify a preferred method of communication with user 101 using interaction device 317. For example, user 101 may customize epidermal electronics device 100 using a mobile communications device in communication with epidermal electronics device 100. The mobile communications device may provide instructions (e.g., through an application) to epidermal electronics device 100 specifying a particular warning for a particular event. For example, user 101 may specify that a vibration be produced by interaction device 317 (rather that a different warning communication such as a noise or light) in response to control circuit 305 determining that a collision is imminent. Customization may be carried out using control circuit 305 (e.g., instructions received by control circuit 305 and stored in memory 309).

In some embodiments, interaction device 317 functions as a reporter. Using interaction device 317 (and/or RF antenna 319, transceiver circuit 303, control circuit 305, etc.), epidermal electronics device 100 can communicate with user 101. For example, epidermal electronics device 100 may communicate warnings to user 100. As described herein, epidermal electronics device 100 may function as personal radar system. In response to detecting or estimating a collision or imminent collision with an object, epidermal electronics device 100 may communicate a warning to user 101. A waning may include one or more of a vibration, sound, light, or other communication. As an additional example, epidermal electronics device 100 may determine or estimate the best orientation for communicating with a remote device such as a cell tower (e.g., when forwarding a communication from a mobile communications device to a cell tower). Epidermal electronics device 100 may communicate to user 101 using interaction device 317 when epidermal electronics device 100 is in or is nearly in optimal orientation for communication with the remote device (e.g., the RF beam from RF antennas 319 is directed toward the remote device).

A previously discussed herein, epidermal electronics device 100 can include one or more RF antennas 319. RF antennas 319 may be any antenna configured to receive and/or transmit electromagnetic radiation in the radio frequency spectrum. RF antennas 319 can be any antenna structure. For example, RF antennas 319 may be one or more of dipole antennas, loop antennas, near field communications antennas, plate antennas, metamaterial antennas, microstrip antennas, patch antennas, and/or other types of antennas. In further embodiments, RF antennas 319 are graphene antennas. Advantageously, this may allow for high data throughput over short distances and may be used for applications such as multi-dimensional data collection from sensors 315 and/or other sensors (e.g., sensors external to epidermal electronics device 100). Multiple types of antennas may be included in a single epidermal electronics device. RF antennas 319 can be constructed with a material and/or geometry such that RF antennas 319 are flexible. RF antennas 319 can be phased using control circuit 305 and/or transceiver circuit 303. In further embodiments, RF antennas 319 are configured to emit and receive radar waves (e.g., electromagnetic radiation in one or more radar spectrums). In alternative embodiments, RF antennas 319 may be configured to receive and/or transmit electromagnetic waves in alternative spectra.

In some embodiments, epidermal electronics device 100 includes a single RF antenna 319. Control circuit 305 may control a plurality of epidermal electronics devices 100, each having a single RF antenna 319. Control circuit 305 may cause a phased emission of radio waves through this control. For example, control circuit 305 may receive orientation data from the plurality of epidermal electronics devices 100. Using this and/or other information, control circuit 305 can cause the transmission of a command which when received causes the plurality of epidermal electronics device 100 to transmit using each individual RF antenna 319 at a specific time or with a specific phase shift. This may cause the plurality of epidermal electronics devices 100 to act as a single phased array; each epidermal electronics device having a single RF antenna 319.

Referring now to FIG. 3B, epidermal electronics device 100 is shown including electronics module 327. In some embodiments, epidermal electronics device 100 houses large components in electronics module 327 separate from sensors 315 and/or interaction devices 317 in electronics assembly 313. These large components may be located outside of the flexible patch which includes electronics layer 207 and barrier layer 209. Electronics module 327 may hold any or all of power source 301, transceiver circuit 303 and/or control circuit 305. In one embodiment, electronics module 327 is separate from electronics layer 207 (e.g., electronics module 327 may house components outside of electronics assembly 313 and may provide for connection to electronics assembly 313). Electronics module 327 may be a housing containing the above mentioned components. For example, electronics module 327 may be a plastic or polymer housing with access to the components housed within. Electronics module 327 may also be a film or other protective encasement.

In some embodiments, electronics module 327 allows for power source 301, transceiver circuit 303 and/or control circuit 305 to be on a larger scale than if they were within electronics layer 207. For example, power source 301 may be a relatively large battery. Processing circuit 305 may be an integrated circuit or SOC. In some embodiments, electronics module 327 is connected to electronics layer 207 by power connection 323. Electronics module 327 may provide power from power source 301 to components of electronics layer 207 (e.g., sensors, interaction devices, etc.) through power connection 323. In further embodiments, electronics module 327 is also connected to electronics layer 207 by input/output connection 325. Electronics module 327 may be connected to electronics layer 207 and/or electronics assembly 313 by one or more input/output connections 325. This may facilitate the use of additional components (e.g., sensors, interactions devices, etc.). The use of multiple input/output connections 325 may reduce the need, partially or completely, for multiplexing.

In some embodiments, multiple electronics layers 207, each with its own separate barrier layer 209 and substrate layer 205 (e.g., multiple epidermal electronics patches), connect to the same electronics module 327. This may allow for measurement and interaction at multiple points on attachment surface 203 with a single supporting power source 301, transceiver circuit 303, and control circuit 610.

Referring now to FIG. 3C, epidermal electronics device 100 is in communication with a remote device in some embodiments. Epidermal electronics device 100 can communicate with a remote device using one or more of RF antenna 319, transceiver circuit 303, control circuit 305 and/or other hardware included in epidermal electronics device 100. As previously discussed, this communication may be wireless communication (e.g., RF transmissions) using one or more protocols (e.g., WiFi, Bluetooth, etc.).

Remote devices may include any device configured to receive and/or send wireless communication signals. For example, remote devices may include cell tower 329, mobile communications device 335, other epidermal electronics devices 100, and/or other devices located remote from epidermal electronics device 100.

Cell tower 329 may be any networking device for use in cellular voice or data communication. For example, cell tower 329 may be a tower for facilitating cellular telephone calls and/or data access. Cell tower 329 may include transmitter 331, receiver 333, and/or other hardware and/or software for establishing cellular communications with mobile communications devices 335 such as cell phones, tablets, vehicles, and/or other devices configured to communicate using a cellular network.

In some embodiments, epidermal electronics device 100 is configured to send transmission to and receive transmissions from cell tower 329. Epidermal electronics device 100 may receive an RF transmission from cell tower 329 using hardware such as RF antenna 319, transceiver circuit 303, and control circuit 305. Epidermal electronics device 100 may then forward the communication to one or more mobile communications devices 335 (e.g., a cellular phone). Epidermal electronics device 100 may also receive RF transmissions from one or more mobile communications devices 100 and transmit an RF signal to cell tower 329. Epidermal electronics device 100 may act as an antenna for mobile communications device 335 and transmit and receive signals from cell tower 329 and mobile communications device 335. Advantageously, epidermal electronics device 100 may include one or more RF antennas 319 such that the aperture or effective aperture of epidermal electronics device 100 is greater than that of an antenna included within mobile communications device 335. This can provide mobile communications device 335 with more effective communications with cell tower 329. For example, the gain using one or more RF antennas 319 of epidermal electronics device 100 may be larger than that of an antenna included in mobile communications device 335. Epidermal electronics device 100 may also generate a directional beam steered toward cell tower 329 (e.g., by phasing RF antennas 319). This may increase signal quality and decrease the power used to communicate with cell tower 329 in comparison to an antenna included in mobile communications device 335 (e.g., acting as a broadband antenna).

Mobile communications device 335 may be an electronic device which is configured to send and/or receive transmissions wirelessly and/or using a wired connection. Mobile communications device 335 may be a device which wirelessly transmits and receives RF voice and/or data transmissions. For example, mobile communications device 335 may be or include one or more of a cell phone, smart phone, tablet computer, laptop computer, wearable computer (e.g., computer included in or worn as a watch, glasses, or other device), implanted device (e.g., a medical device such as a pacemaker), and/or other communications device. Mobile communications device 335 may include one or more of control circuit 305, processor 307, and/or memory 309. These components may allow mobile communications device 335 to perform the functions previously described as being performed by control circuit 305 of epidermal electronics device 100. For example, control circuit 305 of mobile communications device 335 may determine or estimate the position and/or orientation of epidermal electronics device 100 based on sensors 315 included in epidermal electronics device 100. Mobile communications device 335 may perform calculations, make determinations, estimate, or otherwise manipulate information in order to facilitate the functions of mobile communications device 335 described herein. For example, mobile communications device 335 may use algorithms, programs, and/or other data manipulation techniques.

Mobile communications device 335 may send information to and receive information from epidermal electronics device 100 using one or more communications techniques described herein (e.g., RF transmissions using a Bluetooth protocol). This information may be used to perform the functions of epidermal electronics device 100 described herein. For example, mobile communications device 335 may be used to customize the use of epidermal electronics device 100 (e.g., to specific which interaction devices 317 are used to provide alerts to user 101 and/or otherwise communicate with user 101). Mobile communications device 335 may estimate or determine the position and/or orientation of epidermal electronics device 100, control the transmission or refection of RF signals using RF antennas 319, transceiver circuit 303, and/or control circuit 305 of epidermal electronics device (e.g., phase an RF transmission using a plurality of RF antennas 319), create an image of an object using radar data from epidermal electronics device 100, determine the position, speed or trajectory of an object using radar data from epidermal electronics device 100, and/or perform other functions related to epidermal electronics device 100.

A remote device may perform calculations, run algorithms, process data, control epidermal electronics device 100, and/or otherwise interact with epidermal electronics device 100. In one embodiment, epidermal electronics device 100 does not include a processor. Epidermal electronics device 100 may include control circuit 305, power source 301, and/or transceiver circuit 303 for controlling components of epidermal electronics device 100 based on transmissions received from a remote device. The remote device may control epidermal electronics device 100. Data processing, control of epidermal electronics device, and/or other functions can be performed by the remote device.

In some embodiments, mobile communication device 335 includes one or more input/output devices 337. Input/output devices 337 may include any hardware for receiving inputs from and providing outputs to user 101. For example, input/output devices 337 may be or include a display, touchscreen, keyboard, speaker, vibration motor, etc. Input/output devices 337 may be used to communicate information to user 101 such as warnings (e.g., a collision warning based on the radar functions of epidermal electronics device 100). Input/output devices 337 may also receive information for use by one or more epidermal electronics device 100. For example, input/output devices 337 may receive inputs allowing a user 101 to customize the functions of epidermal electronics device 100 (e.g., how warnings are provided to user 101).

Referring now to FIG. 4, epidermal electronics device 100 is illustrated phasing RF antennas 319 according to one embodiment. Control circuit 305 and/or transceiver circuit 303 may control the emission or transmission of RF signals from RF antennas 319. Epidermal electronics device 100 may phase the emission of RF signals from RF antennas 319. Epidermal electronics device 100 may produce, using phased emission, a plurality of RF signals 403 which create a wavefront directed in a particular direction such as toward an RF receiver (e.g., cell tower 329). RF transmissions 403 may be controlled and emitted by RF antennas 319 such that constructive interference between the plurality of transmissions 403 steer the wavefront towards an intended receiver (e.g., cell tower 329). This allows epidermal electronics device 100 to steer the RF transmissions from RF antennas 319. Advantageously, epidermal electronics device 100 can direct an RF transmission towards an intended receiver such that a narrower beam may be used. This may increase signal quality and/or reduce the power used to communicate with a receiver (e.g., cell tower 329).

In some embodiments, epidermal electronics device 100 phases RF antennas 319 in response to the orientation or deformation of epidermal electronics device. As previously discussed, control circuit 305 may determine or estimate the orientation, location, deformation, or other characteristics of epidermal electronics device 100. Using this information, control circuit 305 may further control the phasing of RF antennas 319 to create a wavefront targeted to reach a specific receiver. In further embodiments, control circuit 305 may determine the orientation of epidermal electronics device 100 and use phasing to compensate for a direction, curve, rotation, shape, or other deformation of epidermal electronics device 100. Advantageously, this may allow epidermal electronics device 100 to transmit RF signals as though epidermal electronics device 100 is a flat plane regardless of the actual state of epidermal electronics device 100 (e.g., curved due to attachment surface 203). For example, epidermal electronics device 100 may be curved due to a curved body part of user 101 (e.g., a curved arm). By phasing RF antennas 319, epidermal electronics device 100 may compensate for the orientation of epidermal electronics device 100.

In some embodiments, epidermal electronic device 100 uses one or more sensors 315 to determine the orientation, curvature, location, deformation or other parameters of epidermal electronics device 100. For example, strain sensors 401 (e.g., arranged in multiple orientations) may be used alone or in conjunction with other sensors such as accelerometers, gyroscopes, inclinometers, etc. For example, the curvature or shape of epidermal electronics device 100 may be determined by detecting the relative positions or orientations between individual RF antennas 319 within epidermal electronics device 100, e.g., by transmitting near-field or far-field signals from one or more RF antennas 319 and receiving them from other RF antennas 319, while detecting time, phase, or polarization shifts between the transmitted and received signals.

In further embodiments, control circuit 305 (either in one or more epidermal electronics device 100 or a remote device) may control the phasing of a plurality of epidermal electronics devices 100 based on their orientations. For example, a system may include a plurality of epidermal electronics devices 100 with each epidermal electronics device including one or more RF antennas 319. In some embodiments, each epidermal electronics device 100 includes only a single RF antenna 319 (e.g., a loop antenna). A single, or alternatively multiple, control circuit 305 may collect orientation, location, deformation, and/or other data from the plurality of epidermal electronics devices. Based on this information, control circuit 305 (e.g., located in mobile communications device 335) can control the plurality of RF antennas 319 included in the plurality of epidermal electronics devices 100 (e.g., one RF antenna 319 per epidermal electronics device 100). Control circuit 305 may jointly phase RF antennas 319 to steer a wavefront of RF transmissions (e.g., towards a receiver or to compensate for deformation or orientation of epidermal electronics devices 100).

Using the techniques described herein, a directional beam may be created which reduces the power used in communicating with a receiver. Additionally, the directional beam may result in improved signal quality. In some embodiments, RF antennas 319 are phased in emergency situations to ensure signal quality and reception of the transmission. In other embodiments, RF antennas 319 are phased for each transmission or a subset of transmissions. In further embodiments, epidermal electronics device 100 and/or a remote device in communication with epidermal electronics device 100 has access to location information for one or more receivers (e.g., cell towers). This may allow for directional beams to be transmitted to a specific receiver location. For example, the position of a receiver may be determined using global positioning system information (e.g., from mobile communications device 335), signal triangulation, measuring of signal strength, and/or other techniques to locate a receiver.

In further embodiments, epidermal electronics device 100 may steer or otherwise direct RF transmissions and/or reception towards a remote device while epidermal electronics device 100 moves relative to the remote device. Control circuit 305 can control the phasing of RF antennas 319 such that radio frequency transmissions from the epidermal electronics device remained steered toward a remote device as the epidermal electronics device moves relative to the remote device. RF antennas 319 may be located on a single epidermal electronics device 100 or a plurality of epidermal electronics devices. Control circuit 305 determines or approximates the position of the remote source using one or more of the techniques described herein (e.g., using global positioning system information). Control circuit 305 may also determine or estimate the position and/or orientation of epidermal electronics device 100 using one or more of the techniques described herein (e.g., using sensors 315 such as accelerometers and gyroscopes). In an embodiment, epidermal device 100 can receive a radiofrequency signal (e.g., using RF antennas 319) from a remote device, determine the phasing of the received signal, and use this incident phasing to choose the phasing (e.g., the inverse of the incident phasing) with which to actively phase RF antennas 319 so as to direct a transmission towards the remote device. In one embodiment, control circuit 305 determines the incident phasing by actively phasing a receiving antenna array (e.g., RF antennas 319) so as to optimize reception of the incident signal.

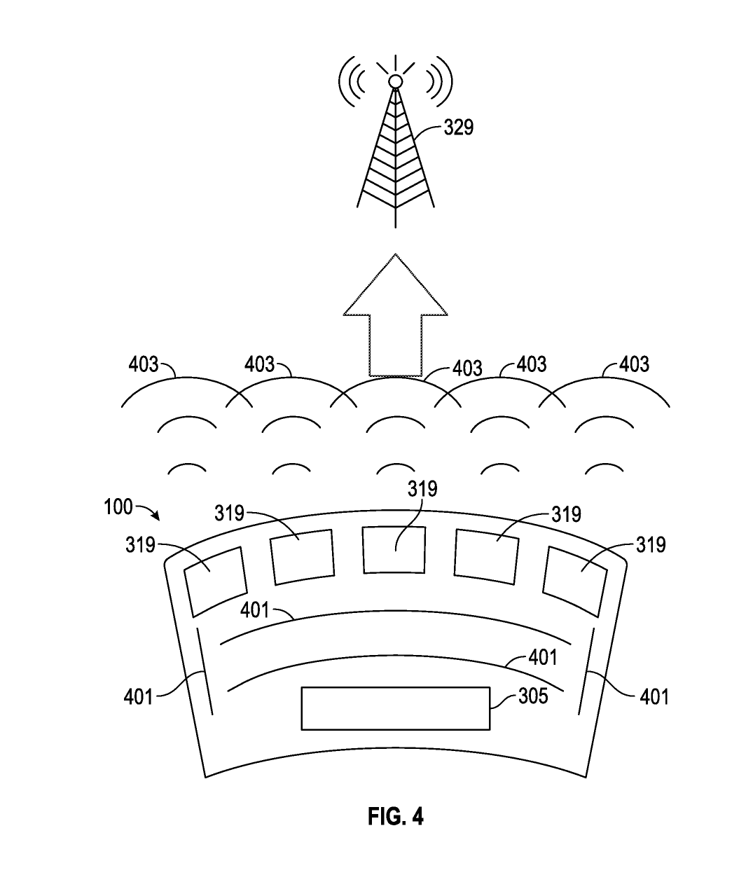

Referring now to FIG. 5, epidermal electronics device 100 is shown functioning as a personal radar according to one embodiment. As previously discussed, epidermal electronics device 100 can emit and receive radar waves using RF antennas 319. Emitted radar waves 503 interact with one or more objects 501 in the environment around epidermal electronics device 100 and user 101. Reflected waves 505 from objects 501 are received at one or more RF antennas 319 at epidermal electronics device 100. Control circuit 305 can use information based on the emitted radar waves 503 and reflected waves 505 to determine information about one or more objects 501. For example, epidermal electronics device 100, using control circuit 305 (e.g., one or more programs, algorithms, and/or other data processing techniques) can estimate and/or determine the location of one or more objects 501, or the speed and/or trajectory of one or more objects 501. Standard radar technique may be used to process reflected waves 505 (e.g., using the time from emission to reception of reflected waves 501, using the Doppler effect to calculate movement of object 501 based on the frequency of reflected waves 501, etc.), track object 501 by phasing RF antennas 319, etc. The radar can utilize any of a variety of radar techniques and waveforms, including narrowband, wideband, or ultra wideband (e.g., micro-impulse radar). Epidermal electronics device 100 acts as a personal radar system for user 101. In some embodiments, epidermal electronics device 100 can transmit radar waves inward, to look at internal organs of user 101. For instance, it can function as a micro-impulse radar to monitor the motion of the user's heart or lungs. Such radar transmissions can be delivered based on a schedule, based on user activity levels, or on sensed physiological conditions (e.g., pulse, blood oxygenation, respiratory sounds, etc.).

In alternative embodiments, epidermal electronics device 100 may function as a passive radar system rather than an active radar system. Epidermal electronics device may receive radar waves without emitting radar waves prior to receiving them. For instance, epidermal electronics device can operate as a radar receiver of a bistatic radar, receiving reflected radar waves emitted from an external radar transmitter. Based on information received from the radar waves, epidermal electronics device 100 may perform one or more of the radar functions described herein.

In alternative embodiments, calculations, determinations, and/or estimations using the above described and/or other radar techniques are performed by control circuit 305 of a remote device. For example, epidermal electronics device 100 may transmit information related to reflected waves 505 (e.g., as determined using transceiver circuit 303) to mobile communications device 335. Mobile communications device 335 may perform calculations to determine one or more properties related to object 501 (e.g., position, direction, speed, trajectory, etc.) using hardware such as control circuit 305 therein.

In some embodiments, a plurality of epidermal electronics devices 100 are used as components within a personal radar system. For example, one or more epidermal electronics devices may transmit radar signals which alternative epidermal electronics devices 100 receive reflected waves 505. A plurality of epidermal electronics devices may be used to measure objects 501 in different locations or fields relative to user 101. For example, one epidermal electronics device 100 may be forward looking and a second epidermal electronics device 100 may be rearward looking. An array of RF antennas 319 may be phased and epidermal electronics device(s) 100 can function as a phased radar array. For example, a plurality of RF antennas 319 on a single epidermal electronics device 100 may be phased to steer an emitted radar beam. Alternatively or additionally, a plurality of epidermal electronics devices 100 may be phased in order to operate as a phased radar array. The phasing of one or more RF antennas 319 and/or one or more epidermal electronics devices 100 may be controlled based on the movement of epidermal electronics device 100 (e.g., posture of user 101, a gesture of user 101 detected by sensors 315 and control circuit 305, etc.). For example, user 101 may perform a gesture in order to indicate that epidermal electronics device 100 should act as a forward collision avoidance radar system. The gesture may be measured and determined to have been made using sensors 315 (e.g., accelerometers, gyroscopes, etc.) and control circuit 305. In response, control circuit 305 may phase RF antennas 319 (on a single or multiple epidermal electronics devices 100) such that emitted radar waves 503 are directed forward from user 101.

In some embodiments, epidermal electronics device 100 performs additional functions using emitted and measured radar waves. For example, epidermal electronics device 100 may create an image of object 501 based on radar returns. Control circuit 305 may apply one or more algorithms or programs to received radar measurements in order to generate a 2-D or 3-D image of object 501. For example, user 101 may travel around object 501 such that radar measurements of the distances to the surface of object 501 are generated. The position of and change in position of epidermal electronics device 100 may be measured using sensors 315 (e.g., accelerometers, gyroscopes, etc.). Using this information control circuit 305 can determine the measurements of object 501 and/or otherwise generate an image of object 501. Other radar based imaging techniques may be used by epidermal electronics device 100 to generate an image of object 100.

In some embodiments, control circuit 305 can emit a radar pulse based upon a schedule, upon a position of user 101, a motion of user 101 (e.g., every time he moves 15 inches), or an ambient light level (e.g., when it is too dark for user 101 to visually see objects). In some embodiments, control circuit 305 can emit a new radar pulse based upon objects detected with a previous radar pulse. For example, the time separation between radar pulses can be based on the proximity or speed of a detected object (e.g., sending radar pulses more frequently for close objects than far ones, or for rapidly approaching objects than for static or departing ones).

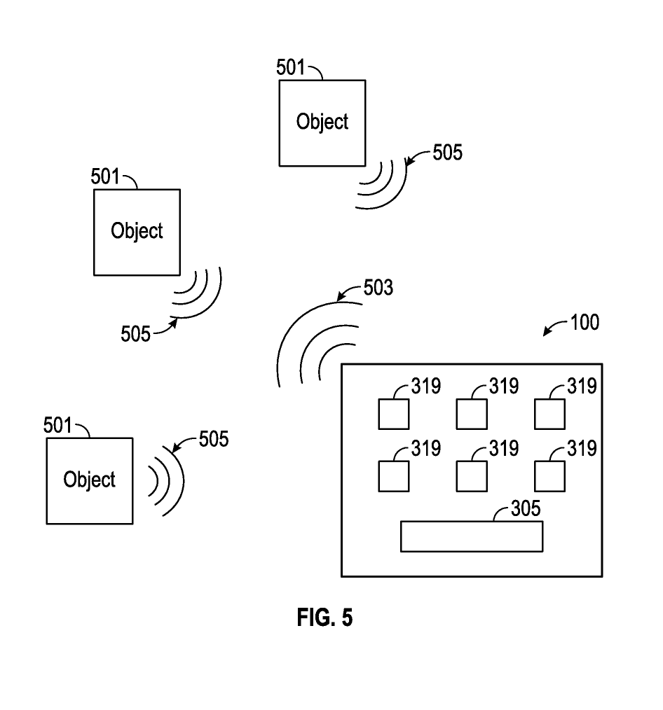

Referring now to FIG. 6, a system of epidermal electronics devices 100 on user 101 is shown according to one embodiment. User 100 may wear a plurality of epidermal electronics devices 100. Multiple electronic devices 100 may communicate with each other and/or a remote device such as mobile communications device 335 and/or cell tower 329. For example, epidermal electronics device 100 may communicate with themselves and/or remote devices using RF antennas 319 and a radio frequency wireless communication protocol such as Bluetooth.

In one embodiment, one or more epidermal electronics devices 100 communicate with mobile communications device 335 and cell tower 329 such that epidermal electronics device 100 functions as an antenna for mobile communications device 335. Epidermal electronics device 100 may have one or more RF antennas 319 which have a greater aperture than that of an antenna included in mobile communications device 335. Epidermal electronics device 100 may receive data and/or signals via a radio wave transmission received from mobile communications device 335. Epidermal electronics device 100 may then transmit the data and/or signal to cell tower 329. The RF antennas 319 of epidermal electronics device 100 can be phased as previously described (e.g., a plurality of antennas on one epidermal electronics device 100 and/or a single or multiple antennas on a plurality of epidermal electronics devices 100). Phasing may be controlled based on the orientation, location, or other parameters of one or more epidermal electronics devices. Phasing may compensate (e.g., approximate a flat antenna) for curvature or other deformation of epidermal electronics device 100. Phasing may also be used to steer a beam to a specific remote device (e.g., cell tower 329).

In further embodiments, epidermal electronics devices 100 function as a personal radar system for user 100. As previously described, epidermal electronics devices 100 may emit radar waves using RF antennas 319. The reflected radar waves may be used to determine one or more properties of object 501 (e.g., a vehicle) such as the location, speed, trajectory, and image of object 501, and/or other information. Epidermal electronics device 100 may communicate information such as an image of object 501, a collision warning, and/or other information to user 101. In some embodiments, epidermal electronics device 100 communicates to user 101 using interaction device 317 (e.g., providing a vibration using a vibration motor). Epidermal electronics device 100 can also communicate with user 101 by providing information to and/or controlling mobile communications device 100. For example, epidermal electronics device 100 may communicate an image of object 501 which is displayed using input/output devices 337 (e.g., displayed on a display of mobile communications device 335). Alternatively, epidermal electronics device 100 can cause mobile communications device 335 to play a noise or vibrate.

In further embodiments, a plurality of epidermal electronics devices 100 can determine the position, location, and/or orientation of each epidermal electronics device 100 relative to the others. Epidermal electronics device 100 and/or a remote device (e.g., mobile communications device 335) can determine the posture of user 101 using this and/or other information. The posture of user 101 can be communicated to mobile communications device 335 and/or other remote devices. The posture of user 101 may be further used to phase one or more RF antennas 319 included in one or more epidermal electronics devices 100.

As shown in FIG. 6, epidermal electronics device 100 may be of varying sizes in alternative embodiments. For example, epidermal electronics device 100 may be approximately the size of the torso of user 100. Advantageously, epidermal electronics device 100 may include larger or a greater number of RF antennas 319 for use in communicating with a remote device. This may improve signal quality, decrease power consumption, and/or allow user 101 to aim an emitted beam towards the remote device and receive transmissions from the remote device. Other sizes of epidermal electronics device 100 are possible.

In further embodiments, epidermal electronics device 100 may perform additional functions other than those previously described herein. For example, epidermal electronics device 100 can function as a volumetric identification device. Epidermal electronics device 100 may identify a particular user 101. This function may be used for security purposes such as preventing unauthorized use of epidermal electronics device 100, transmitting user 101 identification information from epidermal electronics device 100 to a remote device, and/or otherwise providing identification information and/or controlling access based on an identification of user 101. Epidermal electronics device 100 may identify a user using one or more biometric techniques and sensors 315. For example, epidermal electronics device 100 may determine the pattern of hairs on user 101, the pattern of the subsurface blood vessels of user 101, etc. This information may be used by control circuit 305 to identify user 101.

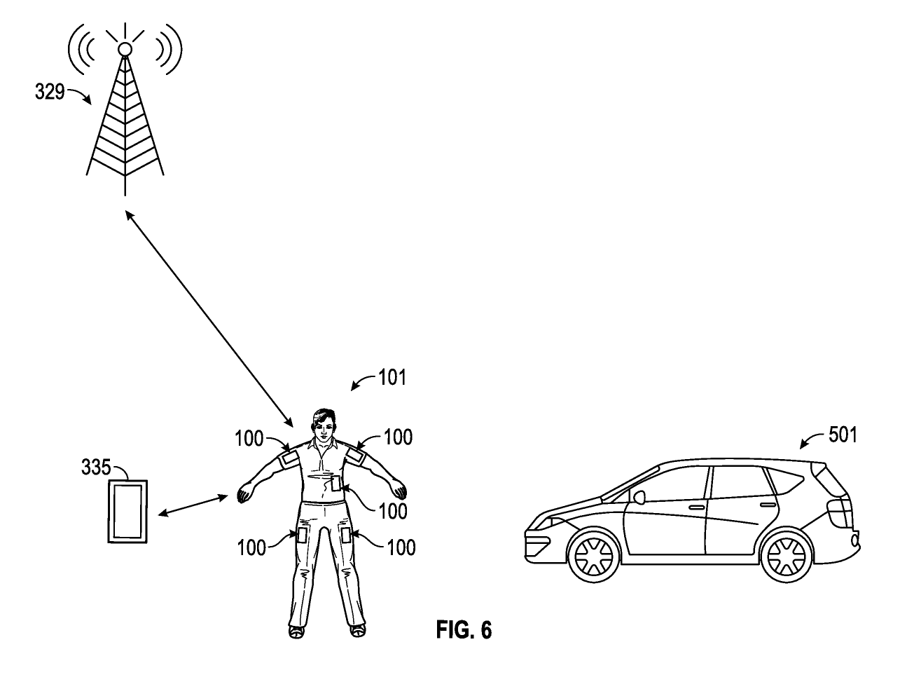

Referring now to FIG. 7, a block diagram for method 700 of communicating with a remote device using epidermal electronics device 100 is illustrated according to one embodiment. Epidermal electronics device 100 can receive data from sensors 315 (701). The data can be related to the position or orientation of epidermal electronics device 100. For example, the data may be from one or more of an accelerometer, inclinometer, and gyroscope. In alternative embodiments, the data from sensors 315 is received using RF antenna 319, and the data is related to the position or orientation of a separate epidermal electronics device 100.

Epidermal electronics device 100 can determine its orientation and/or position (703). Epidermal electronics device 100 can use control circuit 305 to determine its orientation and/or position. In some embodiments, epidermal electronics device 100 determines its position and/or orientation based on locally received data (e.g., from one or more sensors 315 included in epidermal electronics device 100). In alternative embodiments, epidermal electronics device 100 determines its position and/or orientation relative to another epidermal electronics device 100. Epidermal electronics device 100 can receive information from the other epidermal electronics device 100 and use this information (e.g., calculated orientation, calculated position, and/or raw data from sensors 315) to determine its orientation and/or position relative to the other epidermal electronics device 100.

Epidermal electronics device 100 can actively phase RF antennas 319 based on the determined orientation and/or position (705). For example, control circuit 305 can control the timing of emitted transmissions from a plurality of RF antennas 319. The orientation and/or position can be absolute or relative to another epidermal electronics device 100. In some embodiments, this allows epidermal electronics device 100 to approximate a flat transmitter even though epidermal electronics device 100 is curved or otherwise deformed. Additionally, epidermal electronics device 100 can control itself and an additional epidermal electronics device 100 to create a jointly phased array using RF antennas 319 from both epidermal electronics devices 100.

By phasing RF antennas 319, epidermal electronics device 100 can transmit a radio frequency signal (707). For example, the signal transmitted may be a forwarded transmission from mobile communications device 335 to cell tower 329 or another receiver. Advantageously, epidermal electronics device 100 may use one or more RF antennas 319 to increase the gain of the transmission to cell tower 329 relative to the gain of an antenna included in mobile communications device 335. In alternative embodiments, the signal may be in the radar spectrum.

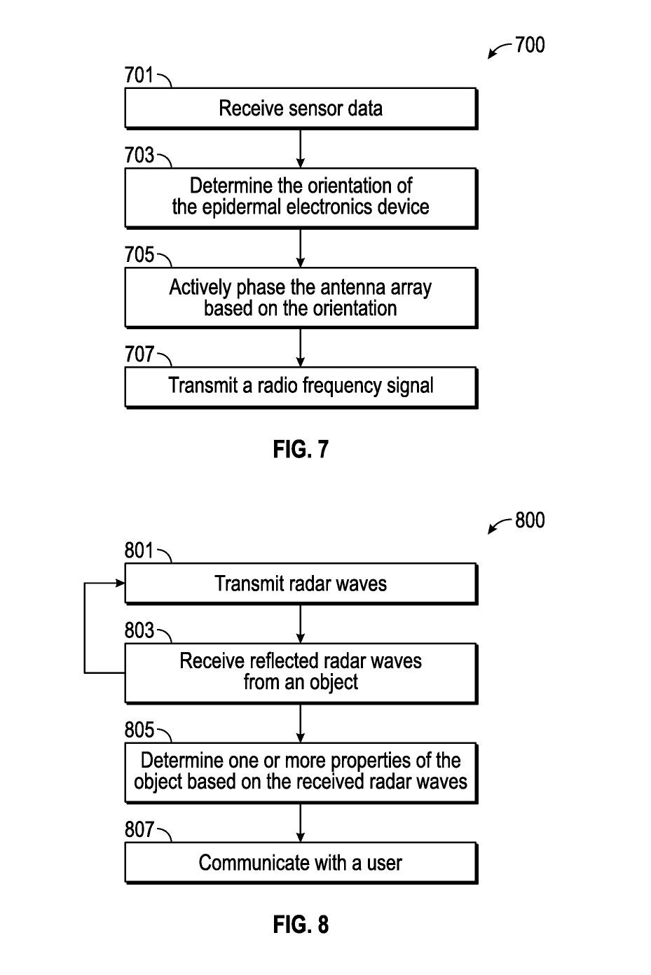

Referring now to FIG. 8, a block diagram of method 800 for performing personal radar functions for user 101 using epidermal electronics device 100 is illustrated according to one embodiment. Epidermal electronics device 100 can transmit radar waves (801). Radar waves can be transmitted using one or more RF antennas 319. In some embodiments, control circuit 305 phases RF antennas 319 such that epidermal electronics device 100 functions as a phased radar array. RF antennas 319 may be phased based on a position and/or an orientation of epidermal electronics device 100, a posture of user 101, a gesture of user 101, ambient light level, and/or other parameters or events. In further embodiments, RF antennas 319 are phased to track object 501 as it moves.

Epidermal electronics device 100 can receive radar waves reflected from object 501 (803). Radar waves which illuminate object 501 can be reflected by object 501. Reflected radar waves from object 501 can be received using one or more RF antennas 319 of epidermal electronics device 100. Radar waves may be transmitted and reflected radar waves may be received iteratively. This may provide epidermal electronics device 100 with information about object 501.

Epidermal electronics device 100 can determine one or more properties of object 501 based on the received radar waves (805). Control circuit 305 can analyze received radar waves using one or more radar processing techniques. Using these techniques, information about object 501 can be determined such as the proximity of object 501 to user 101, the speed of object 501, the trajectory of object 501, if a collision between user 101 and object 501 is likely, and/or other information.

Epidermal electronics device 100 can communicate with user 101 based on information determined about object 501 (807). Epidermal electronics device 100 can communicate with user 101 via one or more interaction devices 317. For example, in response to determining that a collision is likely, epidermal electronics device 100 can produce a vibration with interaction device 317 (e.g., a vibration motor). In alternative embodiments, epidermal electronics device 100 communicates with a user via mobile communications device 335. For example, epidermal electronics device 100 can send a transmission to mobile communications device 335 using RF antennas 319. The transmission can cause mobile communications device 335 to communicate with user 101. For example, mobile communications device 335 can display a message on a screen, play a noise with a speaker, vibrate, and/or otherwise communicate with user 101.

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general purpose computer, special purpose computer, or special purpose processing machines to perform a certain function or group of functions.

Although the figures may show a specific order of method steps, the order of the steps may differ from what is depicted. Also two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. An epidermal electronics device, comprising:
an antenna array coupled to a portion of skin of a user;
a control circuit coupled to the antenna array, wherein the control circuit is configured to transmit and receive radio frequency wireless transmissions via the antenna array, and wherein the control circuit is configured to actively phase the antenna array.

2. (canceled)

3. The epidermal electronics device of claim 1, wherein the control circuit is configured to actively phase the antenna array by applying a time shift between transmission of a signal from one individual antenna of the antenna array and transmission of a signal from another individual antenna of the antenna array.

4. The epidermal electronics device of claim 1, wherein the control circuit is configured to actively phase the antenna array by applying a time shift between delivery of a signal received from one individual antenna of the antenna array and delivery of a signal received from another individual antenna of the antenna array.

5. The epidermal electronics device of claim 1, wherein the control circuit is configured to receive a first radio frequency wireless transmission from a remote device, wherein the control circuit is configured to transmit a second radio frequency wireless transmission based on the received transmission, and wherein the control circuit is configured to direct the second transmission towards the remote device by actively phasing the antenna array.

6-12. (canceled)

13. The epidermal electronics device of claim 1, wherein the control circuit is configured to actively phase the antenna array based on a movement of the epidermal electronics device.

14. The epidermal electronics device of claim 13, wherein the control circuit is configured to actively phase the antenna array such that radio frequency transmissions from the epidermal electronics device remain steered toward a specific point as the epidermal electronics device moves relative to the specific point.

15. The epidermal electronics device of claim 13, wherein the control circuit is configured to actively phase the antenna array such that radio frequency reception from the epidermal electronics device remains steered toward a specific point as the epidermal electronics device moves relative to the specific point.

16. The epidermal electronics device of claim 1, wherein the control circuit is configured to actively phase the antenna array based on an orientation of the epidermal electronics device.

17-20. (canceled)

21. The epidermal electronics device of claim 16, wherein the epidermal electronics device is in wireless communication with a second epidermal electronics device using the antenna array.

22. The epidermal electronics device of claim 21, wherein the control circuit is configured to actively phase the antenna array based on the orientation of the epidermal electronics device relative to the second epidermal electronics device.

23-28. (canceled)

29. The epidermal electronics device of claim 1, wherein the control circuit is configured to actively phase the antenna array such that the antenna array approximates a flat antenna when the epidermal electronics device is deformed.

30. The epidermal electronics device of claim 1, wherein the control circuit is configured to actively phase the antenna array based on a gesture detected by the epidermal electronics device.

31. The epidermal electronics device of claim 30, wherein the control circuit is configured to determine that the gesture has been made based on an input from one or more sensors of the epidermal electronics device.

32-33. (canceled)

34. The epidermal electronics device of claim 1, wherein the control circuit is configured to actively phase the antenna array based on a posture of the user determined by the epidermal electronics device.

35-108. (canceled)

109. An epidermal electronics system for sensing a posture of a user, comprising:
a first epidermal electronics device attached to the skin of the user including:
a first antenna configured to transmit and receive radio frequency wireless signals; and
a first control circuit coupled to the first antenna; and
a second epidermal electronics device attached to the skin of the user including:
a second antenna configured to transmit and receive radio frequency wireless signals; and
a second control circuit coupled to the second antenna,
wherein the first control circuit is configured to estimate at least one of an orientation of the first epidermal electronics device relative to the second epidermal electronics device and a position of the first epidermal electronics device relative to the second epidermal electronics device based on a radio frequency wireless signal received from the second epidermal electronics device using the first antenna.

110. The system of claim 109, wherein the first control circuit is configured to determine the orientation of the first epidermal electronics device relative to the second epidermal electronics device based on a polarization of the received radio frequency wireless signal.

111. The system of claim 109, wherein the first control circuit is configured to determine the position of the first epidermal electronics device relative to the second epidermal electronics device based on a strength of the received radio frequency wireless signal.

112. The system of claim 109, wherein the first antenna comprises an antenna array comprising a first antenna element and a second antenna element.

113. The system of claim 112, wherein the first control circuit is configured to determine the orientation of the first epidermal electronics device relative to the second epidermal electronics device based on comparison of a strength of the radio frequency wireless signal received by the first antenna element and a strength of the radio frequency wireless signal received by the second antenna element.

114-119. (canceled)

120. The system of claim 109, wherein at least one of the first control circuit and the second control circuit determines the posture of the user based on at least one of the orientation of the first epidermal electronics device relative to the second epidermal electronics device and the position of the first epidermal electronics device relative to the second epidermal electronics device.

121-132. (canceled)

133. The system of claim 109, wherein the second control circuit is configured to transmit the signal based on at least one of a time, a time interval from a previous transmission of data, a position of the second epidermal electronics device, an orientation of the second epidermal electronics device, a change in position of the second epidermal electronics device, a change in orientation of the second epidermal electronics device, and a motion of the second epidermal electronics device.

134. The system of claim 109, wherein the second control circuit is configured to transmit the signal based on at least one of a relative position between the first epidermal electronics device and the second epidermal electronics device, a relative orientation between the first epidermal electronics device and the second epidermal electronics device, and a relative motion between the first epidermal electronics device and the second epidermal electronics device.

135-138. (canceled)

139. An epidermal electronics device, comprising:
a barrier layer configured to attach the epidermal electronics device to a portion of skin of a user;
an antenna coupled to the barrier layer; and
a control circuit coupled to the antenna and configured to:
transmit and receive radar waves using the antenna;
control the antenna; and
determine one or more properties of an object based on radar waves received by the antenna.

140. The epidermal electronics device of claim 139, wherein the control circuit is configured to control a second radar wave transmission based on one or more properties of an object determined in response to a first radar wave transmission.

141-144. (canceled)

145. The epidermal electronics device of claim 139, wherein the control circuit is configured to determine a proximity of the object to the user based on received radar waves which were reflected from the object.

146. The epidermal electronics device of claim 139, wherein the control circuit is configured to determine a speed of the object based on received radar waves which were reflected from the object.

147. The epidermal electronics device of claim 139, wherein the control circuit is configured to determine a trajectory of the object based on received radar waves which were reflected from the object.

148-151. (canceled)

152. The epidermal electronics device of claim 139, wherein the control circuit is configured to generate an image of the object based on received radar waves which were reflected from the object.

153. The epidermal electronics device of claim 152, wherein the control circuit is configured to transmit the image of the object to a remote device using the antenna, and wherein the remote device is configured to display the image of the object.

154-156. (canceled)

157. The epidermal electronics device of claim 139, wherein the control circuit is configured to determine if a collision between the user and the object is likely to occur.

158. The epidermal electronics device of claim 157, wherein the control circuit is configured to communicate with the user using an interaction device in response to determining that a collision between the user and the object is likely to occur.

159. The epidermal electronics device of claim 158, wherein the interaction device is included in the epidermal electronics device.

160. (canceled)

161. The epidermal electronics device of claim 158, wherein the control circuit is configured to communicate with the user using by transmitting information to a mobile communications device.

162. The epidermal electronics device of claim 161, wherein the information is configured to cause the mobile communications device to at least one of display a warning message on a display, play a warning noise using a speaker, or produce a vibration using a vibration motor.

163-165. (canceled)

166. The epidermal electronics device of claim 139, wherein the epidermal electronics device is in communication with at least one additional epidermal electronics device using the antenna and radio frequency transmissions, and wherein a system of multiple epidermal electronics devices is configured to act as a personal radar system for the user.

167-202. (canceled)
